(19) United States
(12) Patent Application Publication
(10) Pub. No.: US 2024/0026281 A1
(43) Pub. Date: Jan. 25, 2024

(54) STABILISED LACTIC ACID BACTERIA COMPOSITIONS

(71) Applicant: Chr. Hansen A/S, Hoersholm (DK)

(72) Inventors: Furqan Ali, Mumbai Suburban, Maharashtra (IN); Surender Kumar Dhayal, Hoersholm (DK)

(73) Assignee: Chr. Hansen A/S, Hoersholm (DK)

(21) Appl. No.: 18/254,754

(22) PCT Filed: Nov. 29, 2021
(86) PCT No.: PCT/EP2021/083375

§ 371 Date: May 26, 2023

(30) Foreign Application Priority Data
Nov. 30, 2020 (EP) 20210793.4

(51) Int. Cl. C12N 1/20; C12N 1/04

(52) U.S. Cl. CPC C12N 1/20; C12N 1/04

(57) ABSTRACT

Present invention relates to dried lactic acid bacteria compositions are stabilised with synergistic mixtures of stabilisers selected from oligofructans, maltodextrin, inulin and pea fibre. The mixtures have been found to stabilise the compositions during the drying (e.g. freeze-drying) process and during storage.

The present invention relates to compositions of lactic acid bacteria (LAB) and stabilisers therefor.

Various cultures of bacteria that produce lactic acid and are generally classified as lactic acid bacteria (LAB) are essential in the making of all fermented milk products, cheese and butter. Cultures of such bacteria may be referred to as starter cultures and they impart specific features to various dairy products by performing a number of functions.

Many lactic acid bacteria are known to have probiotic properties (i.e. they have a beneficial health effect on humans and some other animals when ingested). In most cases, it is imperative that the microorganisms remain viable after prolonged storage, in order for them to impart their beneficial effect on ingestion. Attempts have been made in which bacteria that are to be dried (for example freeze-dried) are mixed with additives that can variously: protect cells during a specific step in an industrial process, for example freezing and/or drying; create a matrix to protect cells during shelf life/storage; protect cells in the acidic environment of the stomach and bile; and/or protect cells during rehydration. Protectants that act during the freezing of the cells are termed cryo-protectants. Protectants that act during lyophilisation (freeze-drying) are termed lyo-protectants.

For instance, if the LAB composition is mixed with milk powder to make a suitable infant powder, one generally needs a very storage stable LAB composition, essentially because an infant powder product may be given to infants quite a long time after its actual fabrication date. Accordingly, if the infant powder is given to infants e.g. 30 weeks (or later) after its actual fabrication date, it is evident that the LAB composition incorporated into the infant powder should be quite storage stable in order to maintain viability of the LAB cells.

Bacterial products can alternatively be formulated as frozen products. For example, commercial starter cultures may be distributed as frozen cultures. Highly concentrated frozen cultures, particularly when prepared as pellets, are commercially very useful since such cultures can be inoculated directly into the fermentation medium (e.g. milk or meat) without intermediate transfer. In other words, such highly concentrated frozen cultures comprise bacteria in an amount that makes in-house bulk starter cultures at the end-users superfluous. A “bulk starter” is defined herein as a starter culture propagated at the food processing plant for inoculation into the fermentation medium. Highly concentrated cultures may be referred to as direct vat set (DVS)-cultures. In order to comprise sufficient bacteria to be used as a DVS-culture at the end-users, a concentrated frozen culture generally has to have a weight of at least 50 g and a content of viable bacteria of at least 109 colony forming units (CFU) per gram. WO 2005/080548 (Chr. Hansen) discloses pellet-frozen lactic acid bacteria (LAB) cultures that are stabilised with, for example, a mixture of trehalose and sucrose and do not form clumps when stored.

In prior art processes, a concentrated bacterial culture is obtained by known methods of culturing the bacteria in a growth medium and then concentrating the culture, for example by centrifugation, with the bacteria being separated from the growth medium. The concentrated culture is then admixed with the desired preservative(s) and, shortly thereafter, the resulting mixture is subjected to: freezing; drying, such as freeze-drying, spray-drying or fluidized bed-drying; or freezing followed by freeze-drying.

Carvalho et al (2004) Biotechnol Prog. 20, 248-254 discusses the effects of various sugars added to growth and drying media upon thermotolerance and survival throughout storage of freeze-dried Lactobacillus delbrueckii ssp. Bulgaricus but does not discuss the use of mixtures of protective compounds.

Nevertheless, sometimes mixtures of protective agents are used. For example, WO 2010/138522 (Advanced Bionutrition Corporation) describes a LAB cell culture composition that is said to be useful for incorporation into an infant powder product.

A preferred composition comprises alginate, inulin, trehalose and hydrolyzed protein (see table 1, paragraph [0094]). WO 2013/001089 (Chr. Hansen) discloses a dry LAB composition comprising trehalose, inulin and casein.

Gisela et al (2014) Food & Nutrition Sci. 5, 1746-1755 discloses a synergistic cryoprotective effect when preparing Lactobacillus plantarum compositions can be achieved with mixtures of milk and sucrose and mixtures of sucrose and trehalose.

Inulin and oligofructose (a subgroup of inulin) have been combined (Niness (1999) J. Nutrition 129, 1402S-1406S) in order to stimulate Bifidus growth but there was no disclosure relating to preservation or lactic acid bacteria.

Su et al (2014) J Chinese Inst Food Sci Tech (11), 56-63 disclosed a mixture of inulin, glutamate and sorbitol as protective agents for freeze-drying Lactobacillus plantarum CGMCC but with no disclosure of any synergy.

Mensink et al (2015) Carbohydrate Polymers 130, 405-419 is a review of inulin, including its shorter chain form oligofructose. It mentions that inulin has been used to stabilise protein-based pharmaceuticals, but there was no mention of stabilising microorganisms. Higher MW inulins were said to be useful for storage stability in food products. It was said that inulin has a synergistic effect on gelation with other gelling agents, such as gelatin, alginate, maltodextrins and starch, but there was no mention of synergy in the context of its preservative action.

In Jeong et al (2015) Korean Soc. Biotech. Bioeng. J. 30, 109-113, mixtures of cryoprotectants were tested for Lactobacillus plantarum and Lactococcus lactis, including a mixture of skim milk (or maltodextrin), trehalose, glycerine and sodium chloride. No synergistic effect was disclosed.

In Shu et al (2017) Emirates J. Food & Ag. 29, 256-263 cryoprotectants for Streptococcus thermophilus during freeze-drying were tested. It was said that a better effect could be achieved with the mixture of several protectants, in particular sucrose and soluble starch, plus ascorbic acid as an antioxidant.

Finally, WO 2013/001089 (Chr. Hansen; Yde & Svendsen) disclosed a combination of inulin, trehalose and casein for protection of LAB during freeze-drying. Synergy was not mentioned or shown.

It has now been found that various mixtures of specific agents surprisingly provide a synergistic stabilising effect.

### SUMMARY OF THE INVENTION

A first aspect of the invention provides a dry composition (for example, a freeze-dried or spray-dried formulation) comprising lactic acid bacteria (LAB) and a stabiliser comprising a synergistic mixture of at least a first protectant and a different second protectant, the first and second protectants being selected from the group consisting of oligofructans, maltodextrin, inulin and pea fibre.

Preferably, the first protectant and second protectant are present in the mixture at a ratio of between 5:95 and 95:5, preferably between 10:90 and 90:10, between 20:80 and 80:20, between 30:70 and 70:30, between 40:60 and 60:40, between 45:55 and 55:45, or about 50:50.

The stabiliser can additionally comprise pectin, preferably at a level that is 2-4% of the amount of the first and second protectants combined.

The stabiliser may, for example, comprise a mixture of:
inulin and maltodextrin;
oligofructans and maltodextrin;
oligofructans, maltodextrin and pectin;
inulin, maltodextrin and pectin; or
inulin, maltodextrin and pea fibre.

The industrially most useful lactic acid bacteria are found among Lactococcus species, Streptococcus species, Enterococcus species, Lactobacillus species (including all those that were classed as Lactobacillus until 2020), Leuconostoc species, Bifidobacterium species, Propioni and Pediococcus species. Accordingly, in a preferred embodiment the lactic acid bacteria are selected from the group consisting of these lactic acid bacteria.

The lactic acid bacteria are preferably of a genus selected from the group consisting of Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus. In particular, they can be Limosilactobacillus reuteri, Lacticaseibacillus rhamnosus, Ligilactobacillus salivarius, Lacticaseibacillus casei, Lacticaseibacillus paracasei subsp. Paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, and/or Lactiplantibacillus pentosus. Others include Lactococcus lactis subsp. lactis, Lactococcus lactis subsp. cremoris, Leuconostoc lactis, Leuconostoc mesenteroides subsp. cremoris, Pediococcus pentosaceus, Lactococcus lactis subsp. lactis biovar. diacetylactis, Streptococcus thermophilus, Enterococcus, such as Enterococcus faecum, Bifidobacterium animalis, Bifidobacterium lactis, Bifidobacterium longum, Lactobacillus helveticus, Lactobacillus fermentum, Lactobacillus salivarius, Lactobacillus delbrueckii subsp. bulgaricus and Lactobacillus acidophilus.

The composition may comprise one or more strains of lactic acid bacteria which may be selected from the group comprising: BB-12® (Bifidobacterium animalis subsp. lactis BB-12®), DSM 15954; ATCC 29682, ATCC 27536, DSM 13692, DSM 10140, LA-5 (Lactobacillus acidophilus LA-5®), DSM 13241, LGG® (Lactobacillus rhamnosus LGG®), ATCC 53103, GR-1® (Lactobacillus rhamnosus GR-1®), ATCC 55826, RC-14® (Lactobacillus reuteri RC-14®), ATCC 55845, L. casei 431® (Lactobacillus paracasei subsp. paracasei L. casei 431®), ATCC 55544, F19® (Lactobacillus paracasei F19®), LMG-17806, TH-4® (Streptococcus thermophilus TH-4®), DSM 15957, PCC® (Lactobacillus fermentum PCC®), NM02/31074, and LP-33® (Lactobacillus paracasei subsp. paracasei LP-33®), CCTCC M204012.

The LAB culture may be a “mixed lactic acid bacteria (LAB) culture” or a “pure lactic acid bacteria (LAB) culture”. The term “mixed lactic acid bacteria (LAB) culture”, or “LAB” culture, denotes a mixed culture that comprises two or more different LAB species. The term a “pure lactic acid bacteria (LAB) culture” denotes a pure culture that comprises only a single LAB species. Accordingly, in a preferred embodiment the LAB culture is a LAB culture selected from the group consisting of these cultures.

The LAB culture may be washed, or non-washed, before mixing with the protective agents.

Preferably, the LAB cell is a probiotic cell.

The composition preferably also comprises an antioxidant, such as ascorbic acid or citric acid or a salt of either, for example trisodium citrate, or Vitamin E.

The maximum water content is preferably 5% by weight, and more preferably no more than 3% or 1% by weight.

The composition can contain a weight ratio of LAB to stabiliser mixture (plus antioxidant, if present) that is from about 0.5:1 to 1:40. Preferably, however, the composition comprises 20-50% of the stabiliser (more preferably 30-50%, or 40-50%), 1-25% of the antioxidant (more preferably 5-20%, or 8-15%) and 45-55% LAB (more preferably 49-50%), all percentages being expressed relative to the total content of stabiliser, antioxidant and LAB, plus up to 3% water (preferably no more than 1%) also expressed relative to the total content of stabiliser, antioxidant and LAB.

The composition preferably comprises a content of viable LABs in the range from 10⁸-10¹² CFU/g, preferably 109-10¹² CFU/g, more preferably at least 10¹¹ cfu/g and still more preferably at least 5.0×10¹¹ cfu/g of formulation. The composition may have those values after 8 weeks of storage at 37° C. and a_{w}≤0.15.

A second aspect of the invention provides the use of the stabiliser to stabilise lactic acid bacteria in a dried formulation (for example, a freeze-dried or spray-dried formulation) or in a process for the preparation of a dried formulation (for example, a freeze-dried or spray-dried formulation). The stabiliser provides synergistic cryoprotection, synergistic lyoprotection and/or synergistic storage stability.

A third aspect of the invention provides a method of preparing a LAB composition comprising the steps of (i) formulating lactic acid bacteria in a medium containing a stabiliser as defined above to form a pre-drying composition and (ii) drying the pre-drying composition, for example by spray drying, vacuum drying, air drying, freeze drying, tray drying or vacuum tray drying.

After 8 weeks of storage at 37° C. and a_{w}≤0.15, the viable LAB content is suitably in the range from 10⁸-10¹² CFU/g, preferably 109-10¹² CFU/g, preferably at least 10¹¹ cfu/g and more preferably at least 5.0E+11 cfu/g of formulation.

The LAB composition can be used to manufacture a human food, beverage, probiotic, animal feed, pharmacological or plant health product.

### DESCRIPTION OF THE FIGURES

FIG. 1 shows the effect of individual ingredients along with trisodium citrate (5%, w/w) on the viability of Ligilactobacillus animalis (LA51) in FD-granulates.

FIG. 2 shows the effect of individual stabilisers and combinations of stabilisers combined with trisodium citrate (5%, w/w) on the viability of Ligilactobacillus animalis (LA51) in FD-granulates.

FIG. 3 shows a comparison of viability of LA51 in compositions of the invention with a benchmark composition after 4 weeks (4 W) or 8 weeks (8 W) of storage at a_{w}≤0.15, T=37° C.

FIG. 4 shows the results of a 16 week stability trial at 37° C. and a_{w}≤0.15 of a composition of the invention (stabilised with a mixture of 11.85% oligofructans, 11.85% maltodextrin and 0.3% pectin) compared with compositions respectively containing 24% oligofructans, 24% maltodextrin and 0.3% pectin.

FIG. 5 is a graph showing effect of single stabiliser and mixture of stabilisers on the accelerated storage stability (37° C., aw≤0.15, 12 weeks) of Ligilactobacillus animalis DSM 33570.

FIG. 6 is a graph showing effect of single stabiliser and mixture of stabilisers on the accelerated storage stability (37° C., aw≤0.15, 12 weeks) of Bifidobacterium animalis subsp. Lactis DSM 15954.

FIG. 7 is a graph showing effect of single stabiliser and mixture of stabilisers on the accelerated storage stability (37° C., aw≤0.15, 12 weeks) of Lactococcus lactis subsp. animalis DSM 21404.

FIG. 8 is a graph showing effect of single stabiliser and mixture of stabilisers on the accelerated storage stability (37° C., aw≤0.15, 12 weeks) of Streptococcus thermophilus DSM 15957.

### DESCRIPTION OF PREFERRED EMBODIMENTS

### Definitions

Synergy is defined as a level of stability that is greater than the additive effect of the two protectants used at the same concentration. For example, if a 24% concentration of a mixture of the two protectants provides greater stability (in terms of cryoprotection, lyoprotection and/or storage stability) than the average stability provided by a 24% concentration of the first protectant and by a 24% concentration of the second protectant, then synergy is observed. The values can be plotted as an isobologram on a graph, with, on the X-axis, the effect of the first protectant only (0% to 100% effect), and on the Y-axis the same with the second protectant. If the experimental point for the combination of the protectants is above the line then there is synergy.

Cryoprotection. Frozen biomass of the cells may be added to a stabilizer(s) and antioxidant (trisodium citrate) and mixed at 10° C. until the frozen biomass is liquified in the matrix using a tube revolver/rotator (ThermoFisher Scientific) and mixed for 2 h. The resulting formulations containing the cells are filled in a sterile pipette and added dropwise into liquid nitrogen to form pellets (referred to as ‘PFD’, for pre-freeze-drying) and then stored at −80° C. prior to freeze drying. These PFD are tested for cfu. Protection from the freezing shock to the bacteria is termed cryoprotection.

Lyoprotection. The pre-freeze-drying pellets (PFDs) may be dried using a freeze dryer (Martin Christ, GmbH) using a safe profile (0.3 mbar, 32° C. for a time of 26 hours). After freeze drying, freeze dried granulates (referred to as ‘FD granulates’) are tested for colony forming units (CFU/g). The viability protection from the freeze-drying stresses is termed lyoprotection.

Storage stability can be determined by analysing how the count of viable microbial cells develops over time. Viability of the microbial culture is measured by determining the CFU/g as described herein. Thus, a measure of the storage stability of the microencapsulated microbial culture may be determined by evaluating CFU/g of the dry granulates of microencapsulated microbial culture at time point 0 (just after drying) and after 4 weeks of storage at accelerated storage conditions. In brief, the storage stability of FD granulates or FD ground powder is investigated as follows: a sample of FD granulates of microbial culture (60 mesh ground powder) is blended in CaCO₃ to achieve a sample with a water activity (aw) of 0.15. The sample is placed in an aluminium bag and the bag is sealed so that no air is trapped within it. The bag is stored at 37° C. for 4 weeks and the CFU/g is determined for the sample.

Inulin is a heterogeneous collection of fructose polymers. It consists of chain-terminating glucosyl moieties and a repetitive fructosyl moiety, linked by β(2,1) bonds. The degree of polymerization (DP) of standard inulin ranges from 2 to 60. After removing the fractions with DP lower than 10 during manufacturing process, the remaining product is high-performance inulin. Fractions with DP lower than 10 are regarded herein as oligofructans (see below), not inulin. The average degree of polymerisation of inulin in the context of this invention can be 20-22 or ≥23. The inulin can be employed in the invention in various forms, for example granules and powders, which are commercially available.

Oligofructans (also known as fructo-oligosaccharides (FOS) or oligofructose, and sometimes abbreviated herein to OF) are mixtures of oligosaccharide fructans. FOS can be produced by degradation of inulin, or polyfructose, a polymer of D-fructose residues linked by β(2→1) bonds with a terminal α(1→2) linked D-glucose. The degree of polymerization of inulin ranges from 10 to 60. Inulin can be degraded enzymatically or chemically to a mixture of oligosaccharides with the general structure Glu-Fruₙ (abbrev. GFₙ) and Fruₘ (Fₘ), with n and m ranging from 1 to 7. This process also occurs to some extent in nature, and these oligosaccharides can be found in a large number of plants, especially in Jerusalem artichoke, chicory and the blue agave plant. The main components of commercial products are kestose (GF₂), nystose (GF₃), fructosylnystose (GF₄), bifurcose (GF₃), inulobiose (F₂), inulotriose (F₃), and inulotetraose (F₄). The second class of FOS is prepared by the transfructosylation action of a β-fructosidase of Aspergillus niger or Aspergillus on sucrose. The resulting mixture has the general formula of GFₙ, with n ranging from 1 to 5. Contrary to the inulin-derived FOS, as well as β(1→2) binding, other linkages do occur, however in limited numbers. In this patent application, “FOS” and cognate terms are used to describe the second class of FOS.

Maltodextrin is a polysaccharide that consists of D-glucose units connected in chains of variable length. The glucose units are primarily linked with α(1→4) glycosidic bonds. Maltodextrin is typically composed of a mixture of chains that vary from three to 17 glucose units long. Maltodextrins are classified by DE (dextrose equivalent) and have a DE between 3 and 20, preferably between 10 and 20. The higher the DE value, the shorter the glucose chains, the higher the sweetness, the higher the solubility, and the lower the heat resistance.

Antioxidant. The term “antioxidant” refers to a compound that inhibits oxidation. The antioxidant may be an industrial chemical or a natural compound. As used herein, antioxidants include, but are not limited to, citric acid, vitamin C, vitamin E and glutathione, and derivatives thereof, especially salts such as trisodium citrate and sodium ascorbate. The term ‘vitamin E’ is to be understood as including any and all variants of tocopherols and tocotrienols (alpha, beta, gamma, delta), whether used alone or together.

CFU/g. In the Examples, the stability of a sample is often assessed by counting the colony-forming units (CFU) per gram, using the following assay. Viable cell counts are determined in freeze-dried granulates sampled immediately after freeze-drying and at selected time points during the stability studies. A standard pour-plating method is used. The freeze-dried material is suspended in sterile peptone saline diluent and homogenized by stomaching. After 30 minutes of revitalization, stomaching is repeated and the cell suspension is serially diluted in peptone saline diluent. The dilutions are plated in duplicates on MRS agar (BD Difco™ Lactobacilli MRS Agar, Fisher Scientific). The agar plates are incubated anaerobically for three days at 37° C. Plates with 30-300 colonies are chosen for counting of colony forming units (CFU). The result is reported as average CFU/g freeze-dried sample, calculated from the duplicates.

A global method for enumeration of Lactobacillus acidophilus La-5 is as described below.

Water activity (a_{w}) is a well-known parameter and is the partial vapor pressure of water in a substance divided by the standard state partial vapor pressure of water.

Herein, the standard state is defined as the partial vapor pressure of pure water at the same temperature. Using this definition, pure distilled water has a water activity of exactly one. Suitable methods for measuring a_{w} are set out on the FDA website under the heading “Water Activity (aw) in Foods”, as published on 27 Aug. 2014.

Culturability: cells are culturable if they form a colony on nutrient media that normally support growth of the bacterial species in question.

The term probiotic cell designates a class of cells which is defined as a microbial food or feed supplement which beneficially affects the host human or animal by improving its gastrointestinal microbial balance. The known beneficial effects include improvement of the colonization resistance against the harmful microflora due to oxygen consumption and acid production of the probiotic organisms.

In the present context, the expression “lactic acid bacteria” (LAB)” designates a group of Gram positive, catalase negative, non-motile, microaerophilic or anaerobic bacteria that ferment sugar with the production of acids including lactic acid as the predominantly produced acid, acetic acid, formic acid and propionic acid. The industrially most useful lactic acid bacteria are found among Lactococcus species (spp.), Streptococcus spp., Lactobacillus spp. (including the species that were classified as Lactobacillus until the 2020 taxonomic revision—see below), Leuconostoc spp., Pediococcus spp., Brevibacterium spp., Enterococcus spp. and Propionibacterium spp. Additionally, lactic acid producing bacteria belonging to the group of the strict anaerobic bacteria, bifidobacteria, i.e. Bifidobacterium spp. which are frequently used as food starter cultures alone or in combination with lactic acid bacteria, are generally included in the group of lactic acid bacteria. Even certain bacteria of the species Staphylococcus (e.g. S. carnosus, S. equorum, S. sciuri, S. vitulinus and S. xylosus) have been referred to as LAB (Mogensen et al. (2002) Bulletin of the IDF No. 377, 10-19).

The nomenclature of lactic acid bacteria (LAB) was changed recently; see Zheng et al., Int. J. Syst. Evol. Microbiol. DOI 10.1099/ijsem.0.004107. The change can be summarized as follows:

| Old Name | New Name |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei subsp. | Lacticaseibacillus paracasei subsp. |
| paracasei | Paracasei |
| Lactobacillus plantarum subsp. | Lactiplantibacillus plantarum subsp. |
| plantarum | Plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

The new names will be used in this specification. The LAB can be any of these species.

Commonly used LAB starter culture strains of lactic acid bacteria are generally divided into mesophilic organisms having optimum growth temperatures at about 30° C. and thermophilic organisms having optimum growth temperatures in the range of about 40 to about 45° C.

A composition as described herein may be included in a suitable package—e.g. a bottle, box, vial, capsule etc. As would be understood by the skilled person in the present context, when we refer to the weight of the composition (e.g. termed “g of the composition”) then we are referring to the weight of the composition as such—i.e. not including the possible weight of a suitable package.

General Disclosure Relevant to the Invention

A typical freeze-drying process is performed as follows.
(a) fermenting the LAB cells and harvesting the cells to get a LAB cell concentrate comprising the LAB cells and water; the concentrate can comprises from 10⁸ to 10¹⁴ cfu/g dry matter of the concentrate of lactic acid bacteria (LAB) cells; (b) mixing a suitable amount of the stabiliser mixture with the LAB cell concentrate to form a slurry;
(c) freezing the slurry to form solid frozen particles/pellets;
(d) loading a tray with, for example, from 2 kg/m² to 50 kg/m² of the frozen particles/pellets;
(e) primary drying the material on the tray under a vacuum pressure of, for example, from 0.7 to 2 millibar (mbar), at a temperature wherein the temperature of the material does not get so high that more than 75% of the LAB cells are inactivated and for a period of time until at least 90% of the water of the slurry of step (b) has been removed; and
(f) secondary drying the material of step (e) under a vacuum pressure of, for example, from 0.01 to 0.6 millibar (mbar), at a temperature wherein the temperature of the material does not get so high that more than 75% of the LAB cells are inactivated and for a period of time sufficient to reduce the water activity (a_{w}) to less than 0.30 and thereby obtaining a dry composition of the invention.

Other storage stabilisers and/or lyoprotective agents and/or cryoprotective agents can be included in the composition as well as the synergistic mixtures of the invention. For example, modified starch can be included. However, it is preferred to rely on the defined mixtures of the invention.

The skilled person understands what a dry composition is in the present context. To describe this quantitatively—the water activity (aw) of dry powder composition as described herein is less than 0.30 More preferably, the water activity (a_{w}) of dry powder composition as described herein is less than 0.25, even more preferably less than 0.20 and most preferably the water activity (a_{w}) of dry powder composition as described herein is less than 0.15.

The manufacture of the dry composition as described herein typically involves mixing a cell culture with a protective agent. The second step involves drying said mixture. The drying may be done by freeze drying, spray drying, modified spray drying and/or vacuum drying. Other means for drying may be possible.

In the case of freeze- or vacuum-drying, the mixture is preferably formed into pellets by methods which are known in the art. One method is to let drops of the mixture fall into liquid nitrogen. Another method for forming pellets is by extrusion. The pellets may subsequently be dried, using the above drying methods. Preferably, the composition is dried using the method for preparing a dry powder composition described herein.

The dry composition may be in a powder form.

The weight of the dry composition (e.g. termed “g of the composition”) will generally depend on different factors such as the use of the composition (e.g. to make an infant powder product as discussed below). The weight of the dry composition as described herein may for example be from 1 g to 1000 kg. For instance, if the dry composition is to be used as an infant product, then the dry composition is generally mixed with milk powder and other supplements to obtain an infant powder product comprising lactic acid bacteria cells.

Production of infant powder products may be done on a quite large scale, for example by mixing from 1 to 10 kg of a dry composition as described herein with a suitable amount of milk powder and other supplements.

Accordingly, it is preferred that the weight of the dry composition as described herein is from 50 g to 10000 kg, such as from 100 g to 1000 kg or from 1 kg to 5000 kg or from 100 kg to 1000 kg.

In order to obtain a dry powder composition with a weight of, for example, 100 kg, one needs to use corresponding relatively high amounts of LAB cell concentrate and protective agent(s).

By the term “infant” is meant a human from about birth to 12 months of age. The term “infant formula” refers to a composition in liquid or powdered form that satisfies the nutrient requirements of an infant by being a substitute for human milk. These formulations are regulated by EU and US regulations which define macronutrient, vitamin, mineral and other ingredient levels in an effort to simulate the nutritional and other properties of human breast milk. Evidently, the formula should not contain any potentially allergizing substances. Thus, when hydrolyzed casein is used, it should preferably be hydrolyzed so that over 90% of the peptides have a molecular weight of less than 1,000 Daltons, with over 97% having a molecular weight of less than 2,000 Daltons.

As used herein, “children” are defined as humans over the age of about 12 months to about 12 years old. The infant powder compositions of the present invention may be used for infant formula, follow-on formula, growing up milk and special formula as well as for infant and children's nutritional products for improving their gut microflora while simultaneously providing nutrition to the infant or child.

The dry powder composition of the invention may be encapsulated, for example in a gelatine capsule, or formulated into tablets, or sachets. This aspect is particularly relevant if the composition is to be used in a dietary supplement

If the composition comprises a salt of alginic acid such as sodium alginate, it is often necessary to wash the cells with demineralized water before the addition of the protective agents to avoid the formation of calcium alginate.

The dry composition as described herein may comprise further compounds of interest. This may for example be vitamins (e.g. tocopherol) or other compounds one could be interested in having present in the final composition. Examples of such compounds may be moisture scavengers such as e.g. potato starch.

Depending on the method of drying that is used, it may be necessary to add a viscosity modifier. If, for example, vacuum belt drying is intended, it may be necessary to increase the viscosity. Conversely, if spray drying is intended, it may be necessary to decrease the viscosity. Suitable examples of viscosity modifiers are e.g. water (for decreasing viscosity), pectin, pre-gelatinized starch, gums (e.g. acacia, xanthan, guar gum, locust bean gum), glycerols (e.g. glycerine); glycols (e.g. polyethylene glycols, propylene glycols); plant-derived waxes (e.g. carnauba, rice, candililla), non-plant waxes (beeswax); lecithin; plant fibers; lipids; and silicas (e.g. silicon dioxide).

The dry composition of the invention can be used with or without formulation to form a manufactured product to be given to a human, a mammal, a bird or a fish for health-promoting purposes. This is generally most relevant when the LAB is a probiotic LAB.

A preferred formulation of the invention is in the form of an infant powder, whereby the composition is mixed with milk powder. As known in the art, the milk powder may also comprise other supplements. Another use relates to using the composition as described herein in cereals, such as muesli, or other dry foodstuff.

Accordingly, in further aspects, the invention relates to a food product, such as a cereal, muesli bars, candy bars or chocolate bars, which incorporates the composition according to the invention. It may also be used in powders (e.g. so-called sports powders) intended to be mixed in beverages, such as sport drinks or energy drinks.

In another aspect, the invention relates to a dietary supplement comprising a dry composition as described herein.

It is routine work for the skilled person to ferment a LAB cell of interest in order to e.g. produce/grow it in large scale. As known in the art, harvesting of fermented cells generally involves a centrifugation step to remove relevant parts of the fermentation media and thereby get a LAB cell concentrate. For production of LAB cells one may at this stage have a LAB cell concentrate with around 10% dry matter of cells—i.e. a so-called 10% concentrate. The rest of the concentrate is then normally mainly water, i.e. there will be around 90% of water. The LAB cell concentrate may of course also sometimes contain less water, for example around 50% water. Normally, the LAB cell comprises at least 10% (such as at least 20% or at least 50%) of water. In some embodiments, the concentrate may comprise even less than 10% dry matter, such as in the range of 5-10%, e.g. about 5%.

It is essentially this water of the LAB cell concentrate that is removed by the drying method as described herein to obtain the herein described dry powder LAB composition.

After the harvesting of the cell, it may be preferred to include an extra washing step in order to remove as many of the fermentation media components/compounds as such, to get a more “pure” LAB cell concentrate that essentially only comprises the LAB cells.

### Example 1—Single Stabilisers

In order to investigate and develop a new and improved cryo-formulation, single ingredients, along with trisodium citrate which acts as an antioxidant in the formulation, were tested with Ligilactobacillus animalis (referred to as LA51). Frozen biomass of LA51 was added to the cryo-additives and mixed for 2 hours at 10° C. using a tube Revolver/Rotator (ThermoFisher Scientific) to mimic the production conditions at a manufacturing plant. The resulting composition had an encapsulation index (EI; ratio of stabiliser to bacteria w/w) of 1 on a dry basis.

The formulation containing LA51 was pelletized in the liquid nitrogen and then stored at −80° C. prior to freeze drying. The frozen pellets prior to freeze-drying are referred to as PFD (pre-freeze-drying). The PFDs were dried using a freeze drier (Martin Christ GmbH) following an optimized profile (0.3 mbar and 32° C.). After freeze-drying, freeze-dried granulates (FD granulates) were tested for water activity (a_{w}) and colony-forming units (cfu/g), and subjected to flow cytometry (FlowCyto) analysis for total cells per gram, active cells per gram, active cells/total cells (%) and cfu/active culturability (%). FD granulates packed in aluminium bags with the maximum cfu/g and active/total (%) were tested for storage stability (Temperature=37° C.; a_{w}≤0.15). Based on the maximum cfu/g and active/total (%), these ingredients were formulated in combinations with an encapsulated index of 1 with bacteria on a dry basis.

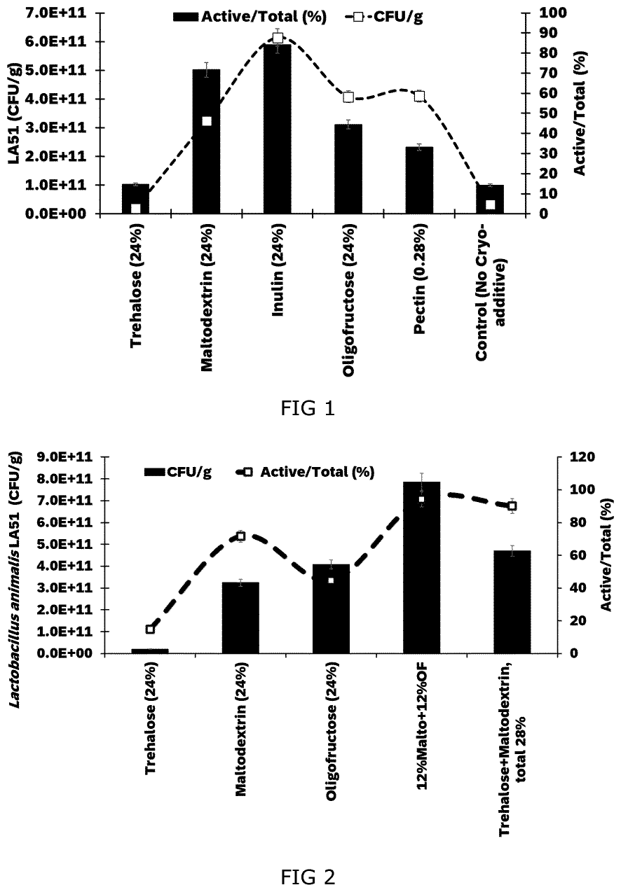

FIG. 1 shows the cfu results and FlowCyto analysis (active/total, %) of FD granulates of single ingredients along with trisodium citrate. The results clearly demonstrate that for the control, without addition of cryo-additives (only trisodium citrate, 5% w/w), the viability of LA51 was drastically reduced (active/total was 14.22% and 3.25E+10 cfu/g) after freeze drying. This indicated the need for cryo- and lyo-protective ingredient addition to the cells of LA51 to protect them from the harsh conditions of manufacturing. The addition of inulin and oligofructose exhibited cryo- and lyo-protective activity with 84.17% and 44.48% active cells and 6.14E+11 and 4.08E+11 cfu/g, respectively, which was significantly higher than the control, thereby suggesting the cryo- and lyo-protective activity. However, trehalose alone did not confer any cryo- and lyo-protection on the LA51 cells, compared with the control.

### Example 2—Mixtures of Stabilisers

Based on the initial cfu and flow cytometry results in Example 1 of single ingredients along with antioxidant (trisodium citrate), inulin and oligofructose showed lyo/cryo activity but had a limited ability to protect bacteria during freeze-drying. Therefore, combinations of more than one ingredient along with antioxidant were carried out to determine the synergistic activity and also to achieve maximum protection of LA51 viability during freeze-drying. As a benchmark, Ligilactobacillus animalis (referred to as LA51) was stabilised with a mixture of trehalose and maltodextrin (together comprising 28% of the composition), including also trisodium citrate as an antioxidant. The results are shown in Table 1 and FIG. 2.

TABLE 1
Viability of LA51 after freezing and freeze-drying, i.e. in FD-granulates.

| | Samples (Composition, w/w) + | | | Active/ | % |
|---|---|---|---|---|---|
| No. | 5% Trisodium citrate | CFU/g | Active/g | total (%) | Culturability |
| 1 | 12% Inulin HSI + 12% | 8.83E+11 | 1.08E+12 | 90.11 | 81.76 |
| | Maltodextrin | | | | |
| 2 | 12% Inulin GR + 12% | 7.17E+11 | 9.30E+11 | 90.07 | 77.10 |
| | Maltodextrin | | | | |
| 3 | 12% Oligofructose + 12% | 7.86E+11 | 1.02E+12 | 94.17 | 77.05 |
| | Maltodextrin | | | | |
| 4 | 11.62% Inulin HSI + 11.62% | 9.03E+11 | 9.75E+11 | 91.44 | 92.62 |
| | Maltodextrin + 0.75% pectin | | | | |
| 5 | 11.62% Inulin GR + 11.62% | 7.23E+11 | 8.77E+11 | 91.06 | 82.44 |
| | Maltodextrin + 0.75% pectin | | | | |
| 6 | 10% Inulin + 10% Maltodextrin + | 6.37E+11 | 7.96E+11 | 88.41 | 80.03 |
| | 4% Pea Fiber | | | | |
| 7 | 11.62% Oligofructan + 11.62% | 8.76E+11 | 1.05E+12 | 92.16 | 83.43 |
| | Maltodextrin + 0.75% pectin | | | | |
| 8 | 10% Oligofructose + 10% | 7.27E+11 | 8.79E+11 | 93.47 | 82.71 |
| | Maltodextrin + 4% Pea Fiber | | | | |
| 9 | Oligofructose + Maltodextrin + | 5.50E+11 | 1.06E+12 | 95.63 | 51.92 |
| | Trehalose | | | | |
| 10 | 10% Inulin + 10% Maltodextrin + | 8.17E+11 | 9.46E+11 | 91.74 | 86.36 |
| | 4% Pea Fiber | | | | |
| 11 | 8% Pea Fiber + 16% Maltodextrin | 5.40E+11 | 9.48E+11 | 92.72 | 56.96 |
| 12 | 8% Pea Fiber + 15.25% | 7.70E+11 | 1.02E+12 | 92.90 | 75.49 |
| | Maltodextrin + 0.75% pectin | | | | |
| 13 | 4% Pea Fiber + 10% Maltodextrin + | 5.60E+11 | 7.86E+11 | 86.65 | 71.25 |
| | 10% Trehalose | | | | |
| 14 | 12% Skimmed milk + 12% whey | 6.57E+11 | 1.01E+11 | 92.07 | 65.05 |
| | sweet | | | | |
| 15 | 11.62% Skimmed milk + 11.62% | 8.47E+11 | 9.78E+11 | 91.37 | 86.61 |
| | whey sweet + 0.75% pectin | | | | |
| 16 | Control (no cryo-additive) | 3.25E+10 | 1.46E+11 | 1.03E+12 | 22.30 |

Inulin HSI and inulin GR were obtained from BENEO GmbH, Mannheim, Germany. Maltodextrin was obtained from Roquette Frères.

The data presented in FIG. 2 and Table 1 clearly show that, when oligofructose and maltodextrin were combined at a 1:1 ratio (dry basis) along with antioxidant and added to LA53 cells (EI=1), the viability (7.86E+11 cfu/g and 94.17 active/total) was significantly higher than single ingredients. The results of this study suggested that two or more of the stabilisers in combination could help achieve maximum viability (˜95%) after freezing and freeze drying (Table 2).

### Example 3—Storage Stability Under Accelerated Conditions

In order to evaluate the storage stability of bacteria using the synergistic stabilisers according to the invention, after freeze drying, freeze-dried (FD) granulates prepared in accordance with Example 2 were packed in aluminium bags and stored at 37° C. They were then tested for CFU at 0, 4 and 8 weeks. The mixture of trehalose and maltodextrin was used as the benchmark in this Example, as in Example 2.

TABLE 2
Storage stability results (CFU/g) of LA51 FD granulate
at 37° C. with water activity (a_{w}) ≤0.15.

| | | LA51 FD granulate (CFU/g) | | |
|---|---|---|---|---|
| No. | Cryo-Formulation | 0 Week | 4 Weeks | 8 Weeks |
| 1 | Trehalose + Maltodextrin | 5.65E+11 | 2.55E+10 | 2.55E+09 |
| 2 | OF + Maltodextrin | 7.86E+11 | 9.70E+10 | 7.90E+09 |
| 3 | OF + Maltodextrin + Pectin | 8.16E+11 | 1.14E+11 | 3.11E+10 |
| 4 | OF + Maltodextrin + Pea fibre | 8.73E+11 | 7.60E+10 | 8.10E+09 |
| 5 | OF + Trehalose + Maltodextrin | 5.50E+11 | 4.40E+10 | 9.04E+09 |
| 6 | Trehalose + OF + Pea fibre | 7.63E+11 | 3.30E+10 | 8.50E+09 |
| 7 | Inulin + Maltodextrin | 8.73E+11 | 4.90E+10 | 7.30E+09 |
| 8 | Inulin + Maltodextrin + Pectin | 8.26E+11 | 8.95E+10 | 1.07E+10 |
| 9 | Inulin + Maltodextrin + Pea fibre | 8.56E+11 | 8.95E+10 | 1.59E+10 |
| 10 | Pea fibre + Maltodextrin | 6.47E+11 | 1.29E+10 | 3.00E+09 |
| 11 | Pea fibre + Maltodextrin + pectin | 6.40E+11 | 3.09E+10 | 3.30E+09 |
| 12 | Trehalose + Maltodextrin + Pea fibre | 6.60E+11 | 4.40E+10 | 4.75E+09 |
| 13 | Skim milk + whey sweet | 8.03E+11 | 9.30E+10 | 1.36E+10 |
| 14 | Skim milk + whey sweet + Pectin | 7.06E+11 | 8.30E+10 | 9.70E+09 |
| 15 | Control (No cryo-additives) | 3.25E+10 | 4.85E+08 | 4.67E+07 |

OF = oligofructans

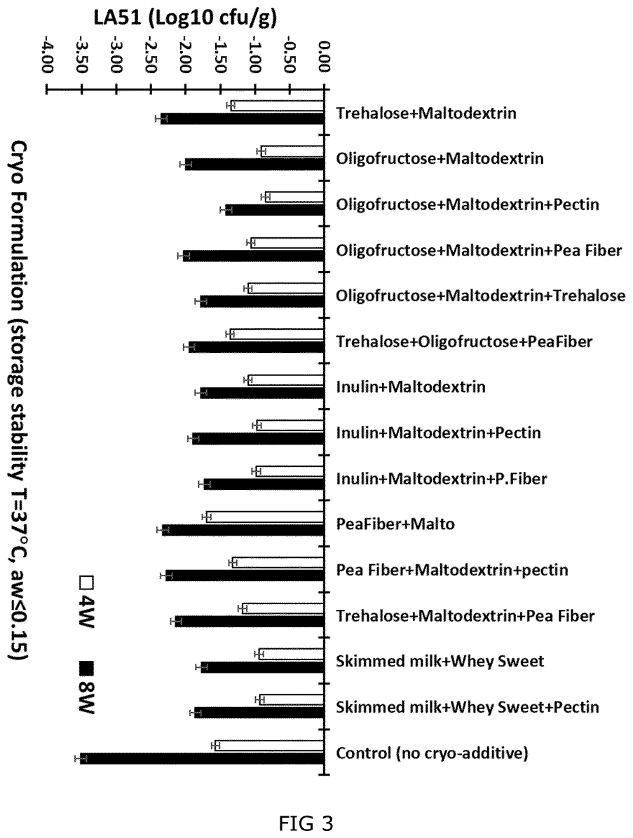

The data presented in FIG. 3 and Table 2 show that synergistic formulations in accordance with the invention have better viability protection than the benchmark mixture of trehalose and maltodextrin after 4 weeks and also after 8 weeks of storage at 37° C. There was 0.85 Log cfu/g reduction of LA51 in cryo-formulation containing oligofructose, maltodextrin and pectin, whereas in case of the comparator it was 1.35 Log cfu/g reduction. Furthermore, this formulation showed higher viability protection (1.42 Log cfu/g reduction) after 8 weeks compared with the benchmark (2.35 Log cfu/g reduction).

### Example 4—Demonstration of Synergy

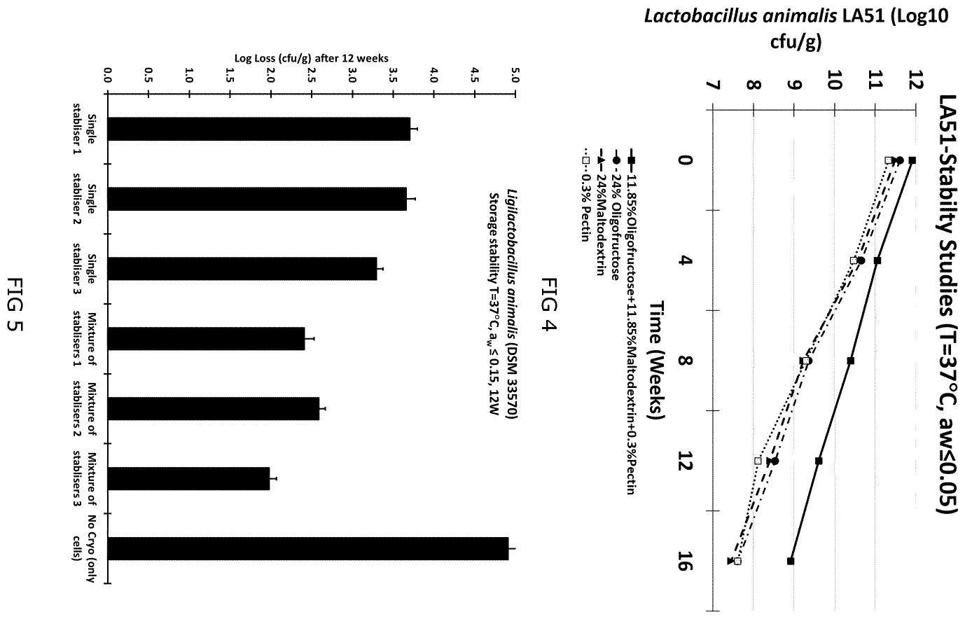

In order to evaluate the synergy of stabilizers during storage stability, single stabilizers (oligofructose, maltodextrin, pectin) were prepared as described in Example 1 and multiple stabilizer (oligofructose, maltodextrin, pectin, together) were prepared as described in Example 2. Storage stability was evaluated as described in Example 3. FIG. 4 represented the Log₁₀ cfu/g of Ligilactobacillus animalis LA51 during storage at 37° C. and a_{w}≤0.15. The data clearly demonstrated that single stabilizers had lesser viability protection compared to multiple stabilizers mixed. The viability protection of synergistic stabilizers was evident as multiple stabilizers showed more than 1 Log₁₀ cfu/g protection than single stabilizers after 16 W of storage at 37° C. and a_{w}≤0.15.

### Example 5—Further Demonstration of Synergy

Bifidobacterium animalis lactis BB12 CHCC 5445 was inoculated into a De Man, Rogosa and Sharpe (MRS) liquid medium (BD Difco™ Lactobacilli MRS Agar, Fisher Scientific) supplemented with 0.5 g/L of L-cysteine hydrochloride (Sigma-Aldrich, Inc.) and cultured anaerobically at 37° C. for 24 hours. After the 24 hours of growth, cells in the medium were concentrated 25 fold using centrifugation. The concentrated cells were mixed with cryoprotectants according to Table 3. Similar to this, Ligilactobacillus animalis LA51 CHCC 10506, Streptococcus thermophilus TH4 CHCC 2336 and Lactococcus lactis R607 CHCC 1915 were grown in De Man, Rogosa and Sharpe (MRS) liquid medium anaerobically at 37° C. for 24 hours. These concentrated cells were also mixed with cryoprotectants according to Table 3.

TABLE 3
The composition of cryoprotectants (% w/w).

| | Ingre- | | | | | | | | |
|---|---|---|---|---|---|---|---|---|---|
| No. | dients | CP-1 | CP-2 | CP-3 | CP-4 | CP-5 | CP-6 | CP-7 | CP-8 |
| 1 | Malto- | 12 | 12 | | | 24 | | 11.85 | 11.85 |
| | dextrin | | | | | | | | |
| | DE12 | | | | | | | | |
| 2 | Oligo- | | 12 | | 24 | | | 11.85 | |
| | fructose | | | | | | | | |
| 3 | Inulin | 12 | | 24 | | | | | 11.85 |
| 4 | Pectin | | | | | | 4 | 0.3 | 0.3 |
| 5 | Ascor- | 6 | 6 | 6 | 6 | 6 | 6 | 6 | 6 |
| | bate | | | | | | | | |
| 6 | RO | 70 | 70 | 70 | 70 | 70 | 70 | 70 | 70 |
| | water | | | | | | | | |

The stability of a sample is assessed by counting the colony-forming units (CFU) per gram, using the following assay. Viable cell counts are determined in freeze-dried granulates sampled immediately after freeze-drying and at selected time points during the stability studies. A standard pour-plating method is used. The freeze-dried material is suspended in sterile peptone saline diluent (BD Difco™ Lactobacilli MRS Agar, Fisher Scientific) and homogenized by stomaching using stomacher (bio\acute{M}rieux, Inc. Durham, NC). After 30 minutes of revitalization, stomaching is repeated and the cell suspension is serially diluted in peptone saline diluent. For the cfu of Bifidobacterium animalis lactis BB12 CHCC 5445, the dilutions are plated in duplicates on MRS agar (BD Difco™ Lactobacilli MRS Agar, Fisher Scientific) supplemented with 0.5 g/L of L-cysteine hydrochloride (Sigma-Aldrich, Inc.). The agar plates are incubated anaerobically for three days at 37° C. For the cfu of Ligilactobacillus animalis La51 CHCC 10506, the dilutions are plated in duplicates on MRS agar (BD Difco™ Lactobacilli MRS Agar, Fisher Scientific). The agar plates are incubated anaerobically for three days at 37° C. In case of Streptococcus thermophilus TH4 CHCC 2336 cfu, the dilutions are plated in duplicates on M17 agar (BD Difco™ Lactobacilli MRS Agar, Fisher Scientific) supplemented with 0.5 g/L of monosodium phosphate (Sigma-Aldrich, Inc.) and 0.5 g/L of disodium phosphate (Sigma-Aldrich, Inc.). The agar plates are incubated aerobically for three days at 37° C. For the cfu of Lactococcus lactis R607 CHCC 1915, the dilutions are plated in duplicates on M17 agar (BD Difco™ Lactobacilli MRS Agar, Fisher Scientific) supplemented with 0.5 g/L of monosodium phosphate (Sigma-Aldrich, Inc.) and 0.5 g/L of disodium phosphate (Sigma-Aldrich, Inc.). The agar plates are incubated aerobically for three days at 37° C. Plates with 30-300 colonies are chosen for counting of colony forming units (CFU). The result is reported as average CFU/g freeze-dried sample, calculated from the duplicates.

The results are shown in Tables 4-7. Table 4 shows storage stability results (CFU/g) of Ligilactobacillus animalis (DSM 33570) at the accelerated conditions (37° C. with ≤0.15). Table 5 shows storage stability results (CFU/g) of Bifidobacterium animalis subsp. Lactis (DSM 15954) at the accelerated conditions (37° C. with ≤0.15). Table 6 shows Storage stability results (CFU/g) of Lactococcus lactis subsp. Lactis (DSM 21404) at the accelerated conditions (37° C. with ≤0.15). Table 7 shows Storage stability results (CFU/g) of Streptococcus thermophilus (DSM 15957) at the accelerated conditions (37° C. with ≤0.15).

TABLE 4
Storage stability results (CFU/g) of Ligilactobacillus animalis (DSM
33570) at the accelerated conditions (37° C. with ≤0.15).

| | Sam- | | | | | |
|---|---|---|---|---|---|---|
| | ple | Time (weeks) | | | | |
| No. | IDs | 0 | 2 | 4 | 8 | 12 |
| 1 | CP-1 | 8.83E+11 | 2.10E+11 | 1.12E+11 | 2.31E+11 | 3.43E+09 |
| 2 | CP-2 | 7.86E+11 | 1.10E+11 | 2.02E+11 | 1.21E+11 | 2.03E+09 |
| 3 | CP-3 | 5.03E+11 | 9.80E+10 | 5.57E+10 | 8.29E+09 | 9.88E+07 |
| 4 | CP-4 | 4.07E+11 | 9.20E+10 | 4.57E+10 | 2.29E+09 | 8.88E+07 |
| 5 | CP-5 | 3.09E+11 | 1.50E+11 | 3.72E+10 | 1.70E+09 | 1.57E+08 |
| 6 | CP-6 | 2.14E+11 | 8.90E+10 | 2.95E+10 | 2.00E+09 | 1.18E+07 |
| 7 | CP-7 | 8.76E+11 | 4.00E+11 | 1.15E+11 | 2.51E+10 | 9.13E+09 |
| 8 | CP-8 | 7.24E+11 | 4.30E+11 | 2.55E+11 | 3.51E+10 | 7.63E+09 |
| 9 | CP-9 | 3.25E+10 | 7.80E+09 | 4.85E+08 | 4.67E+07 | 3.98E+05 |

TABLE 5
Storage stability results (CFU/g) of Bifidobacterium animalis
subsp. Lactis (DSM 15954) at the accelerated
conditions (37° C. with ≤0.15).

| | Sam- | | | | | |
|---|---|---|---|---|---|---|
| | ple | Time (weeks) | | | | |
| No. | IDs | 0 | 2 | 4 | 8 | 12 |
| 1 | CP-1 | 1.40E+09 | 6.75E+08 | 2.51E+08 | 1.48E+08 | 1.13E+08 |
| 2 | CP-2 | 1.34E+09 | 8.95E+08 | 1.92E+08 | 1.00E+08 | 6.20E+07 |
| 3 | CP-3 | 2.62E+09 | 2.00E+07 | 1.25E+06 | 1.00E+04 | 2.00E+03 |
| 4 | CP-4 | 2.70E+09 | 2.00E+07 | 2.45E+06 | 1.10E+04 | 1.00E+03 |
| 5 | CP-5 | 1.19E+09 | 4.50E+08 | 8.70E+07 | 3.55E+07 | 1.05E+07 |
| 6 | CP-6 | 3.10E+09 | 2.90E+09 | 3.85E+08 | 7.95E+08 | 2.30E+07 |
| 7 | CP-7 | 1.73E+09 | 6.60E+08 | 2.02E+08 | 3.22E+08 | 1.65E+08 |
| 8 | CP-8 | 8.20E+08 | 6.50E+08 | 1.87E+08 | 1.21E+08 | 1.35E+08 |
| 9 | CP-9 | 2.90E+11 | 1.30E+11 | 1.88E+09 | 8.55E+08 | 1.40E+08 |

TABLE 6
Storage stability results (CFU/g) of Lactococcus lactis subsp. Lactis
(DSM 21404) at the accelerated conditions (37° C. with ≤0.15).

| | Sam- | | | | | |
|---|---|---|---|---|---|---|
| | ple | Time (weeks) | | | | |
| No. | IDs | 0 | 2 | 4 | 8 | 12 |
| 1 | CP-1 | 1.63E+11 | 9.85E+10 | 7.20E+09 | 3.50E+09 | 9.20E+08 |
| 2 | CP-2 | 1.64E+11 | 8.10E+10 | 5.35E+10 | 9.00E+09 | 2.80E+09 |
| 3 | CP-3 | 1.49E+11 | 1.00E+07 | 1.01E+07 | 5.00E+05 | 2.90E+05 |
| 4 | CP-4 | 3.20E+11 | 1.00E+07 | 7.95E+07 | 1.00E+05 | 7.40E+06 |
| 5 | CP-5 | 3.00E+11 | 6.70E+10 | 6.45E+10 | 3.25E+09 | 4.44E+08 |
| 6 | CP-6 | 2.90E+11 | 2.98E+10 | 1.26E+09 | 1.30E+07 | 3.10E+06 |
| 7 | CP-7 | 3.20E+11 | 9.10E+10 | 1.88E+10 | 1.10E+10 | 9.20E+09 |
| 8 | CP-8 | 2.60E+11 | 9.00E+10 | 6.95E+10 | 1.00E+10 | 8.80E+09 |
| 9 | CP-9 | 6.85E+11 | 2.68E+10 | 1.15E+10 | 1.05E+08 | 4.10E+07 |

TABLE 7
Storage stability results (CFU/g) of Streptococcus thermophilus (DSM
15957) at the accelerated conditions (37° C. with ≤0.15).

| | Sam- | | | | | |
|---|---|---|---|---|---|---|
| | ple | Time (weeks) | | | | |
| No. | IDs | 0 | 2 | 4 | 8 | 12 |
| 1 | CP-1 | 3.30E+11 | 6.70E+10 | 8.72E+09 | 7.83E+09 | 5.41E+09 |
| 2 | CP-2 | 4.30E+11 | 5.30E+10 | 9.75E+09 | 9.65E+09 | 5.80E+09 |
| 3 | CP-3 | 4.30E+11 | 1.17E+10 | 2.20E+06 | 5.49E+06 | 1.10E+06 |
| 4 | CP-4 | 4.30E+11 | 2.07E+10 | 1.00E+06 | 1.58E+06 | 1.10E+06 |
| 5 | CP-5 | 2.30E+11 | 5.40E+10 | 5.75E+09 | 1.06E+09 | 1.34E+09 |
| 6 | CP-6 | 4.10E+11 | 6.30E+10 | 3.41E+10 | 1.63E+09 | 5.00E+08 |
| 7 | CP-7 | 3.10E+11 | 7.40E+10 | 4.05E+09 | 9.55E+09 | 4.80E+09 |
| 8 | CP-8 | 2.20E+11 | 4.10E+10 | 3.35E+09 | 7.85E+09 | 3.90E+09 |
| 9 | CP-9 | 1.40E+10 | 1.50E+09 | 2.50E+06 | 1.85E+05 | 1.60E+05 |

Table 8 summarizes various compositions according to embodiments of the invention, as set out in Table 3, for further comparison in FIGS. 5-8.

TABLE 8
Composition of single stabilisers and mixture of stabilisers
for the demonstration of synergy of cryoprotectants.

| No. | Sample IDs | Composition |
|---|---|---|
| 1 (CP-3) | Single stabiliser 1 | 24% Inulin + 6% Ascorbate |
| 2 (CP-4) | Single stabiliser 2 | 24% Oligofructose + 6% Ascorbate |
| 3 (CP-5) | Single stabiliser 3 | 24% Maltodextrin DE12 + 6% Ascorbate |
| 4 (CP-1) | Mixture of | 12% Inulin + 12% Maltodextrin DE12 + |
| | stabilisers 1 | 6% Ascorbate |
| 5 (CP-2) | Mixture of | 12% Oligofructose + 12% Maltodextrin |
| | stabilisers 2 | DE12 + 6% Ascorbate |
| 6 (CP-7) | Mixture of | 11.86% Oligofructose + 11.86% |
| | stabilisers 3 | Maltodextrin DE12 + 0.3% Pectin + |
| | | 6% Ascorbate |
| 7 | No Cryo (only | No Cryo protectant were added to cells- |
| | cells) | Control group |

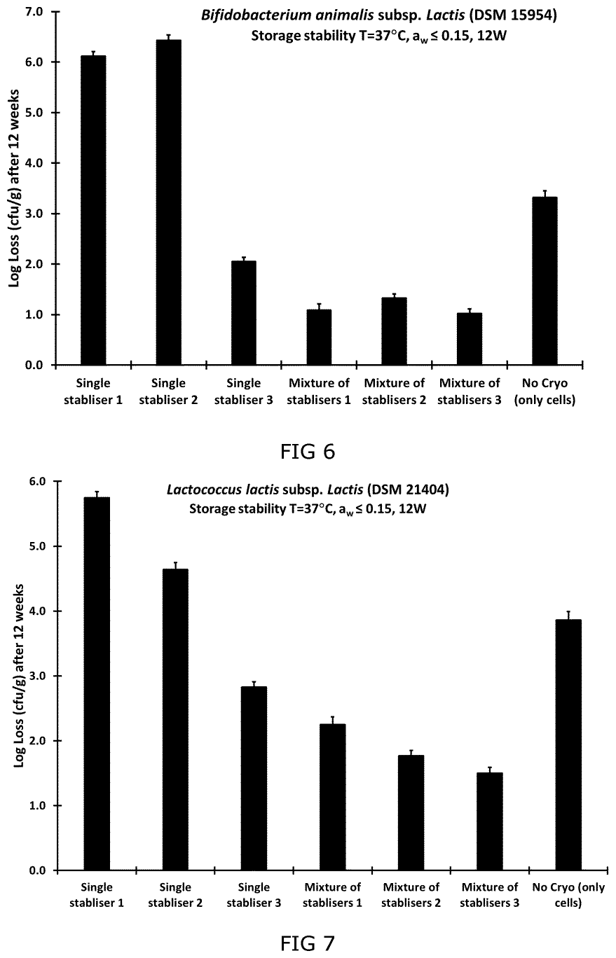

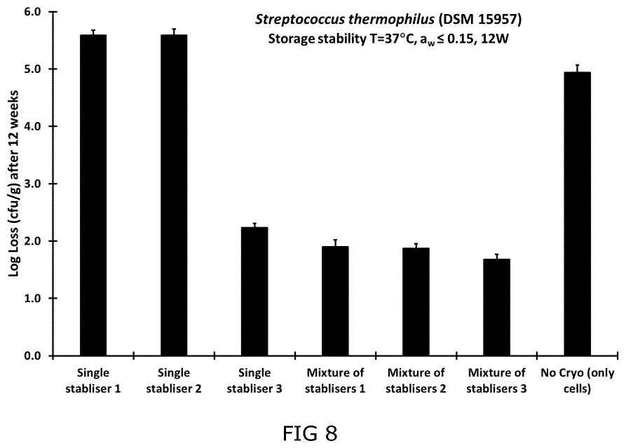

FIG. 5 shows the effect of single stabiliser and mixture of stabilisers on the accelerated storage stability (37° C., aw≤0.15, 12 weeks) for the different cryoprotectants in formulations comprising Ligilactobacillus animalis DSM 33570. FIG. 6 shows effect of single stabiliser and mixture of stabilisers on the accelerated storage stability (37° C., aw≤0.15, 12 weeks) for the different cryoprotectants in formulations comprising Bifidobacterium animalis subsp. Lactis DSM 15954. FIG. 7 shows effect of single stabiliser and mixture of stabilisers on the accelerated storage stability (37° C., aw≤0.15, 12 weeks) for the different cryoprotectants in formulations comprising Lactococcus lactis subsp. animalis DSM 21404. FIG. 8 shows effect of single stabiliser and mixture of stabilisers on the accelerated storage stability (37° C., aw≤0.15, 12 weeks) for the different cryoprotectants in formulations comprising Streptococcus thermophilus DSM 15957.

1. A dry composition (for example, a freeze-dried or spray-dried formulation) comprising lactic acid bacteria (LAB) and a stabiliser comprising a synergistic mixture of at least a first protectant and a different second protectant, the first and second protectants being selected from the group consisting of oligofructans, maltodextrin, inulin and pea fibre.

2. A composition according to claim 1 wherein the first protectant and second protectant are present in the mixture at a ratio of between 5:95 and 95:5, preferably between 10:90 and 90:10, between 20:80 and 80:20, between 30:70 and 70:30, between 40:60 and 60:40, between 45:55 and 55:45, or about 50:50.

3. A composition according to claim 1 or 2 wherein the stabiliser additionally comprises pectin, preferably at a level that is 2-4% of the amount of the first and second protectants combined.

4. A composition according to any of claims 1 to 3 wherein the stabiliser comprises a mixture of:
(i) inulin and maltodextrin;
(ii) oligofructans and maltodextrin;
(iii) oligofructans, maltodextrin and pectin;
(iv) inulin, maltodextrin and pectin; or
(v) inulin, maltodextrin and pea fibre.

5. A composition according to any one of claims 1 to 4 wherein the lactic acid bacteria are of a genus selected from the group consisting of Streptococcus (e.g. Streptococcus thermophilus), Lactococcus (e.g. Lactococcus lactis), Oenococcus (e.g. Oenococcus oeni), Leuconostoc (e.g. Leuconostoc mesenteroides, Leuconostoc pseudomesenteroides), Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus

6. A composition according to claim 5 wherein the bacteria are a species selected from the group consisting of Limosilactobacillus reuteri, Lacticaseibacillus rhamnosus, Ligilactobacillus salivarius, Lacticaseibacillus casei, Lacticaseibacillus paracasei subsp. Paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, Lactiplantibacillus pentosus, Levilactobacillus brevis, Lactobacillus delbrueckii subsp. bulgaricus, Lactobacillus delbrueckii subsp. lactis, Lactobacillus gasseri, Lactobacillus johnsonii, Lactobacillus helveticus and Lactobacillus acidophilus, Lactobacillus jensenii, and Lactobacillus iners.

7. A composition according to any of claims 1 to 6 additionally comprising an antioxidant, such as ascorbic acid or citric acid or a salt of either, for example trisodium citrate, or Vitamin E.

8. A composition according to any of claims 1 to 7 wherein the maximum water content is 5% by weight, preferably no more than 3% or 1% by weight.

9. A composition according to claim 7 or 8 comprising 20-50% of the stabiliser (preferably 30-50%, or 40-50%), 1-25% of the antioxidant (preferably 5-20%, or 8-15%) and 45-55% LAB (preferably 49-50%), all percentages being expressed relative to the total content of stabiliser, antioxidant and LAB, plus up to 3% water (preferably no more than 1%) also expressed relative to the total content of stabiliser, antioxidant and LAB.

10. A composition according to any of claims 1 to 9 comprising a content of viable LABs in the range from 10⁸-10¹² CFU/g, preferably at least 10¹¹ cfu/g and more preferably at least 5.0×10¹¹ cfu/g of formulation.

11. The composition according to claim 10, wherein the composition has been stored 8 weeks at 37° C. and a_{w}≤0.15.

12. The use of a stabiliser as defined in any of claims 1 to 4 to stabilise lactic acid bacteria in a dried formulation (for example, a freeze-dried or spray-dried formulation) or in a process for the preparation of a dried formulation (for example, a freeze-dried or spray-dried formulation).

13. The use according to claim 12 wherein the stabiliser is used to provide synergistic cryoprotection, synergistic lyoprotection and/or synergistic storage stability.

14. A method of preparing a composition according to any of claims 1 to 11 comprising the steps of (i) formulating lactic acid bacteria in a medium containing a stabiliser as defined in any of claims 1 to 4 to form a pre-drying composition and (ii) drying the pre-drying composition.

15. A method according to claim 14 wherein the drying step comprises spray drying, vacuum drying, air drying, freeze drying, tray drying or vacuum tray drying.

16. A method according to claim 14 or 15 wherein the viable LAB content is in the range from 10⁸-10¹² CFU/g, preferably at least 10¹¹ cfu/g and more preferably at least 5.0E+11 cfu/g of formulation.

17. The method according to claim 14, further comprising a step (iii) of storing the composition 8 weeks at 37° C. and a_{w}≤0.15, wherein the viable LAB content is in the range from 10⁸-10¹² CFU/g, preferably at least 10¹¹ cfu/g and more preferably at least 5.0E+11 cfu/g of formulation after storage.

18. A human food, beverage, probiotic, animal feed, pharmacological product or plant health product comprising a composition according to any of claims 1 to 11.
